(19) United States
(12) Patent Application Publication
(10) Pub. No.: US 2024/0127951 A1
(43) Pub. Date: Apr. 18, 2024

(54) METHODS AND SYSTEMS FOR PREDICTING BASELINE CREATININE VALUES

(71) Applicant: KONINKLIJKE PHILIPS N.V., EINDHOVEN (NL)

(72) Inventors: Erina Ghosh, Cambridge, MA (US); Larry Eshelman, Cambridge, MA (US); Stephanie Lanius, Cambridge, MA (US); Emma Holdrich Schwager, Cambridge, MA (US); Kianoush Kashani, Rochester, MN (US)

(21) Appl. No.: 18/275,637

(22) PCT Filed: Jan. 29, 2022
(86) PCT No.: PCT/EP2022/052152

§ 371 Date: Aug. 3, 2023

Related U.S. Application Data
(60) Provisional application No. 63/146,924, filed on Feb. 8, 2021.

(51) Int. Cl. G16H 50/20

(52) U.S. Cl. CPC G16H 50/20

(57) ABSTRACT

A method (100) for determining a baseline creatinine value for a subject, comprising: obtaining (130) a set of features about the subject; analyzing (140), using a trained baseline creatinine determination model, the obtained set of features to generate a baseline creatinine value for the subject; reporting (150), via a user interface, the generated baseline creatinine value for the subject.

### FIELD OF THE INVENTION

The present disclosure is directed generally to methods and systems for determining a baseline creatinine value for a subject.

### BACKGROUND

To identify kidney failure in a patient, creatinine laboratory measurements are compared with the patient's baseline creatinine value. A baseline creatinine value, or the normal creatinine value of a person, can be defined as the mean of all creatinine measurements in the approximately six months to one week before hospitalization of the person. It is an important value to evaluate a patient's baseline renal function and proper dosing of certain medications. However, this value is often unknown at the time of hospitalization. Without knowing the actual baseline creatinine value, it is difficult to determine whether the subject's creatinine value as measured in the hospital is elevated or not. Elevated creatinine, compared to baseline creatinine, is associated with kidney injury, hypertension, diabetes, malnutrition, and other acute conditions.

Since a patient's baseline creatinine value is often unavailable, various empirical equations have been used to estimate baseline creatinine One of the most common methods is the Modification of Diet in Renal Disease study (MDRD) equation. This empirical equation uses age, race and gender as input parameters. Since the MDRD equation is an empirical formula and doesn't consider any chronic conditions, it is often a poor predictor of actual creatinine. Research has suggested, for example, that the MDRD equation underestimates creatinine values for patients with elevated creatinine values and overestimates creatinine values for patients with low creatinine values. This is particularly problematic when computing the risk of acute kidney injury (AKI), which is calculated using urine output and serum creatinine values to classify AKI risk into one of three stages: Stage 1, low risk of AKI; Stage 2, moderate risk of AKI; and Stage 3, severe risk of AKI. Staging based on creatinine values uses a relative increase compared to the patient's respective baseline creatinine value. If a patient's baseline creatinine value is not available, the baseline creatinine value is often estimated using MDRD formula. Thus, if the estimated baseline creatinine value is estimated poorly, this can lead to false positives or false negatives resulting in missed diagnoses or unnecessary interventions.

### SUMMARY OF THE INVENTION

Accordingly, there is a need for methods and systems that more accurately estimate baseline creatinine values for patients.

The present disclosure is directed to inventive methods and systems for determining a baseline creatinine value for a subject. Various embodiments and implementations herein are directed to a baseline creatinine determination system configured to more accurately determine the subject's baseline creatinine value, without having actual measurements of creatinine levels. The system obtains a set of features about the subject, such as from a medical record database or other source of subject data. A trained baseline creatinine determination model of the system analyses the obtained set of features to generate a baseline creatinine value for the subject. The system then reports the generated baseline creatinine value for the subject via a user interface. According to an embodiment, the set of features includes one or more of: (i) chronic kidney disease status; (ii) age of the subject; (iii) weight of the subject; (iv) height of the subject; (v) hypertension status; (vi) nephritis, nephrosis, and/or renal sclerosis status; (vii) Charlson comorbidity index; (viii) weight increase from hospital admission to ICU admission; and/or (ix) history of calcineurin inhibitor intake. According to an embodiment, the methods and systems also comprise a method for training the baseline creatinine determination model of the system.

Generally in one aspect, a method for determining a baseline creatinine value for a subject is provided. The method includes obtaining a set of features about the subject; analyzing, using a trained baseline creatinine determination model, the obtained set of features to generate a baseline creatinine value for the subject; reporting, via a user interface, the generated baseline creatinine value for the subject.

According to an embodiment, the set of features comprises: (i) chronic kidney disease status; (ii) age of the subject; (iii) weight of the subject; (iv) height of the subject; (v) hypertension status; and (vi) nephritis, nephrosis, and/or renal sclerosis status. According to an embodiment, the set of features further comprises: (vii) Charlson comorbidity index; (viii) weight increase from hospital admission to ICU admission; and (ix) history of calcineurin inhibitor intake.

According to an embodiment, the method further includes training the baseline creatinine determination model, comprising: obtaining training data comprising data for a plurality of training subjects, the data comprising: (i) a set of features for each of the training subjects; and (ii) a measured baseline creatinine value for each of the training subjects; training the baseline creatinine determination model using the training dataset to generate a trained baseline creatinine determination model, wherein training comprises identifying a subset of the set of features that generates a generated baseline creatinine value for a training subject that best correlates with the measured baseline creatinine value for that subject, wherein the identified subset of features comprises input features for the trained baseline creatinine determination model; and storing the trained baseline creatinine determination model. According to an embodiment, the set of features for each of the training subjects comprises at least (i) chronic kidney disease status; (ii) age of the training subject; (iii) weight of the training subject; (iv) height of the training subject; (v) hypertension status; and (vi) nephritis, nephrosis, and/or renal sclerosis status.

According to an embodiment, the trained baseline creatinine determination model is a gradient boosting regression model.

According to an embodiment, reporting via the user interface further comprises providing one or more of demographic information about the patient and information about the set of features.

According to an embodiment, the method further includes administering a treatment to the subject based at least in part on the reported generated baseline creatinine value for the subject.

According to another aspect is a system for determining a baseline creatinine value for a subject. The system includes: a set of features about the subject; a trained baseline creatinine determination model; a processor configured to analyze, using the trained baseline creatinine determination model, the obtained set of features to generate a baseline creatinine value for the subject; and a user interface configured to report the generated baseline creatinine value for the subject.

According to an embodiment, the system further includes training data comprising data for a plurality of training subjects, the data comprising: (i) a set of features for each of the training subjects; and (ii) a measured baseline creatinine value for each of the training subjects. According to an embodiment, the processor is further configured to train the baseline creatinine determination model using the training dataset to generate a trained baseline creatinine determination model, wherein training comprises identifying a subset of the set of features that generates a generated baseline creatinine value for a training subject that best correlates with the measured baseline creatinine value for that subject, wherein the identified subset of features comprises input features for the trained baseline creatinine determination model.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for determining a baseline creatinine value for a subject, in accordance with an embodiment.

FIG. 2 is a schematic representation of a baseline creatinine determination system, in accordance with an embodiment.

FIG. 3A is a histogram of error (measured baseline creatinine—predicted) comparing GBR6 and MDRD-60 models for the Mayo Clinic cohort, in accordance with an embodiment.

FIG. 3B is a histogram of error (measured baseline creatinine—predicted) comparing GBR6 and MDRD-60 models for the MIMIC-III cohort, in accordance with an embodiment.

FIG. 4A is a comparison of correlation between measured baseline creatinine and estimated baseline creatinine using the MDRD for sub cohorts of real baseline creatinine for the Mayo Clinic cohort, in accordance with an embodiment.

FIG. 4B is a comparison of correlation between measured baseline creatinine and estimated baseline creatinine using the gradient boosting model for sub cohorts of real baseline creatinine for the Mayo Clinic cohort, in accordance with an embodiment.

FIG. 4C is a comparison of correlation between measured baseline creatinine and estimated baseline creatinine using the MDRD for sub cohorts of real baseline creatinine for the MIMIC-III cohort, in accordance with an embodiment.

FIG. 4D is a comparison of correlation between measured baseline creatinine and estimated baseline creatinine using the gradient boosting model for sub cohorts of real baseline creatinine for the MIMIC-III cohort, in accordance with an embodiment.

FIG. 5A is a histogram of the ratio of estimated and measured baseline creatinine for the GBR6 model vs. the MDRD-60 model for the Mayo Clinic cohort, in accordance with an embodiment.

FIG. 5B is a histogram of the ratio of estimated and measured baseline creatinine for the GBR6 model vs. the MDRD-60 model for the MIMIC-III cohort, in accordance with an embodiment.

FIG. 6A is a correlation plot for Mayo clinic data between a model estimate and actual baseline, in accordance with an embodiment.

FIG. 6B is a correlation plot for MIMIC-III clinic data between a model estimate and actual baseline, in accordance with an embodiment.

FIG. 7A is a graph of feature importance for a GBR6 model based on SHAP (SHapley Additive exPlanations) values, in accordance with an embodiment.

FIG. 7B is a graph of feature importance for a GBR9 model based on SHAP (SHapley Additive exPlanations) values, in accordance with an embodiment.

### DETAILED DESCRIPTION OF EMBODIMENTS

The present disclosure describes various embodiments of a baseline creatinine determination system and method. More generally, Applicant has recognized and appreciated that it would be beneficial to provide a system that more accurately determines a baseline creatinine value for a subject. The system receives or obtains a set of features about the subject, such as from a medical record database or other source of subject data. A trained baseline creatinine determination model of the system analyses the obtained set of features to generate a baseline creatinine value for the subject. The system then reports the generated baseline creatinine value for the subject via a user interface. According to an embodiment, the set of features includes one or more of: (i) chronic kidney disease status; (ii) age of the subject; (iii) weight of the subject; (iv) height of the subject; (v) hypertension status; (vi) nephritis, nephrosis, and/or renal sclerosis status; (vii) Charlson comorbidity index; (viii) weight increase from hospital admission to ICU admission; and/or (ix) history of calcineurin inhibitor intake. According to an embodiment, the methods and systems also comprise a method for training the baseline creatinine determination model of the system.

The baseline creatinine determination systems and methods disclosed or otherwise envisioned herein provides numerous advantages over the prior art. Providing a baseline creatinine determination system or method that more accurately estimates baseline creatinine values for a patient allows for better comparison of baseline levels to measured levels, such as those measured in a care setting such as an ICU, emergency department, hospital, or other care setting. Better comparisons of baseline creatinine levels to measured levels allows for better understanding of the patient's health and improved diagnoses and care. Thus, implementation of the baseline creatinine determination systems and methods disclosed or otherwise envisioned herein improves patient care and outcomes, and potentially saves lives.

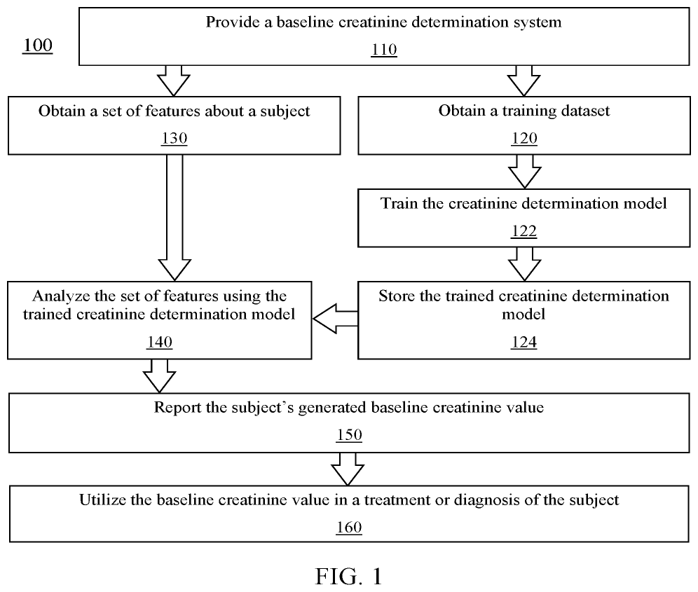

Referring to FIG. 1, in one embodiment, is a flowchart of a method 100 for determining a baseline creatinine value for a subject using a baseline creatinine determination system. The methods described in connection with the figures are provided as examples only, and shall be understood not to limit the scope of the disclosure. The baseline creatinine determination system can be any of the systems described or otherwise envisioned herein. The baseline creatinine determination system can be a single system or multiple different systems.

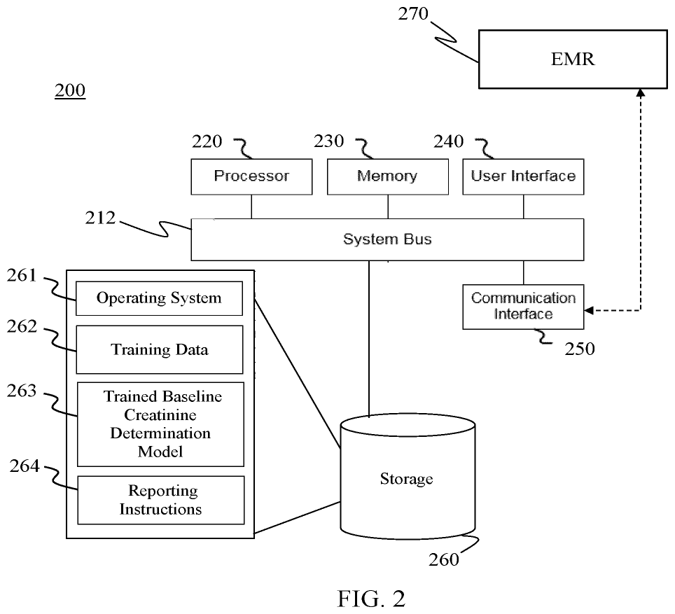

At the end of step 110 of the method, a baseline creatinine determination system 200 is provided. Referring to an embodiment of a baseline creatinine determination system 200 as depicted in FIG. 2, for example, the system comprises one or more of a processor 220, memory 230, user interface 240, communications interface 250, storage 260, and electronic medical record or system (EMR) 270, interconnected via one or more system buses 212. It will be understood that FIG. 2 constitutes, in some respects, an abstraction and that the actual organization of the components of system 200 may be different and more complex than illustrated. Additionally, baseline creatinine determination system 200 can be any of the systems described or otherwise envisioned herein. Other elements and components of system 200 are disclosed and/or envisioned elsewhere herein.

At step 130 of the method, the baseline creatinine determination system receives or obtains a set of features about the subject, where the set of features comprises a plurality of different features or datapoints about the subject.

According to an embodiment, the set of features about the subject is received from, retrieved from, or otherwise obtained from an electronic medical record (EMR) database or system 270. The EMR database or system may be local or remote. The EMR database or system may be a component of the baseline creatinine determination system, or may be in local and/or remote communication with the baseline creatinine determination system. The received patient information may be utilized immediately, or may be stored in local or remote storage for use in further steps of the method.

According to another embodiment, the set of features about the subject is entered into or otherwise provided to the baseline creatinine determination system via a user interface. For example, a user such as a clinician may be prompted by the system to enter one or more datapoints comprise one or more of the set of features. This can be in response to programmed prompts or another system configured to obtain this information from a clinician. For example, the set of features can be input into the system using a user interface via a touchscreen, data entry fields, oral information, or any other method for inputting information into a system.

According to another embodiment, the set of features about the subject comprises a known set of features, some or all of which might be required by the system to make a baseline creatinine value determination as described or otherwise envisioned herein. The set of features may be, for example, patient demographics, clinical information from one or more healthcare information systems including electronic medical records (EMR), radiology information systems, cardiology information systems, lab information systems, and other systems. From these sources, the following types of information that are potentially useful for baseline creatinine determination or estimation can received, obtained, or otherwise collected: (i) patient demographic information (for example age, gender, race, height, weight, etc.); (ii) previous hospital or other care setting visit information (for example time of appointment and visit, reason for visit, healthcare providers, etc.); (ii) current hospital or other care setting clinical information (for example diagnosis, medical imaging and report, treatment, medication, etc.); (iii) medical history and family history; (iv) other information from other sources; and many other types or sources of patient information. According to an embodiment, the baseline creatinine determination system may query an electronic medical record database or system, comprising fast healthcare interoperability resources (FHIR) for example, to obtain the patient information.

According to an embodiment, the set of features about the subject comprises one or more of: (i) chronic kidney disease status; (ii) age of the subject; (iii) weight of the subject; (iv) height of the subject; (v) hypertension status; and (vi) nephritis, nephrosis, and/or renal sclerosis status. According to an embodiment, the set of features about the subject comprises one or more of: (i) chronic kidney disease status; (ii) age of the subject; (iii) weight of the subject; (iv) height of the subject; (v) hypertension status; (vi) nephritis, nephrosis, and/or renal sclerosis status; (vii) Charlson comorbidity index; (viii) weight increase from hospital admission to ICU admission; and (ix) history of calcineurin inhibitor intake. According to an embodiment, the set of features about the subject comprises one or more additional features in addition to any of the features described or otherwise envisioned above.

At step 140 of the method, the baseline creatinine determination system analyzes the obtained set of features using a trained baseline creatinine determination model, to generate a baseline creatinine value for the subject. According to an embodiment, the model can be any model trained to utilize the set of features as input to generate the baseline creatinine determination model. For example, the baseline creatinine determination model may be trained using the methods described or otherwise envisioned herein. According to an embodiment, the trained baseline creatinine determination model is a stored component of the baseline creatinine determination system, or is in local or remote communication with the baseline creatinine determination system.

For example, the baseline creatinine determination model can be trained as described below. At step 120 of the method, a training system—which is the baseline creatinine determination system or another system—obtains a training dataset. The training dataset comprises data for a plurality of training subjects, which is used to train the baseline creatinine determination model. According to an embodiment, the training dataset comprises, for each of a plurality of training subjects, at least: (i) a set of features for each of the training subjects; and (ii) a measured baseline creatinine value for each of the training subjects. Once obtained, the training dataset may be utilized immediately or stored for future use. The training dataset may be obtained from any database or source of data that comprises the necessary information to train the model.

At step 122 of the method, the baseline creatinine determination model is trained using the training dataset, in order to generate a trained baseline creatinine determination model. According to an embodiment, training comprises identifying a subset of the set of features in the training dataset that generates a generated baseline creatinine value for a training subject that best correlates with the measured baseline creatinine value for that subject. According to an embodiment, the identified subset of features comprises input features for the trained baseline creatinine determination model when performing an analysis at step 130 of the method.

At step 124 of the method, the trained baseline creatinine determination model is stored in memory for future analyses. The model may be stored in local or remote memory. According to an embodiment, the trained baseline creatinine determination model is stored in memory of the baseline creatinine determination system.

At step 150 of the method, the baseline creatinine determination system reports the generated baseline creatinine value for the subject via a user interface. The generated baseline creatinine value for the subject can be provided via the user interface using any method for conveying or displaying information, and the user interface can be any device, interface, or mechanism for providing the conveyed or displayed information. For example, the generated baseline creatinine value for the subject may be communicated by wired and/or wireless communication to another device. For example, the system may communicate the information to a mobile phone, computer, laptop, wearable device, and/or any other device configured to allow display and/or other communication of the information. The user interface can be any device or system that allows information to be conveyed and/or received, and may include a display, a mouse, and/or a keyboard for receiving user commands.

According to an embodiment, the baseline creatinine determination system reports the generated baseline creatinine value for the subject via a user interface along with other information. For example, the communicated information may include information about the patient, including but not limited to the identity of the patient, patient demographics, diagnosis or treatment information, and a wide variety of other possible information. The communicated information may include information about the set of features utilized as input to the trained baseline creatinine value model, as well as other information.

At optional step 160 of the method, information provided via the user interface, such as the generated baseline creatinine value for the subject, is utilized by a clinician or a clinical decision support system to diagnose and/or treat the subject. For example, the generated baseline creatinine value for the subject can be utilized when computing the risk of acute kidney injury (AKI). According to an embodiment, AKI is calculated using urine output and serum creatinine values to classify AKI risk into one of three stages: Stage 1, low risk of AKI; Stage 2, moderate risk of AKI; and Stage 3, severe risk of AKI. Staging based on creatinine values uses a relative increase compared to the patient's respective baseline creatinine value, which can be the baseline creatinine value generated for the subject by the baseline creatinine determination system. Once the subject is diagnosed with a risk of acute kidney injury on the basis of the generated baseline creatinine value for the subject, the clinician may implement or administer a treatment configured to prevent acute kidney injury. The treatment may include one or more of hemodialysis, catherization, antibiotics, drugs to increase or decrease fluid retention, and/or many other treatments. For example, in addition to these treatments, a caregiver could modify existing medication orders to remove nephrotoxic drugs. A caregiver could also put a patient on an AKI watchlist, which monitors patients at risk of developing AKI. These are just a few non-limiting examples of treatments that could be implemented or affected by the methods and systems described or otherwise envisioned herein.

Accordingly, the methods and systems described or otherwise envisioned herein provide numerous advantages over the prior art. For example, the system provides a more accurate determination or estimate of a subject's baseline creatinine values, which allows for better comparison of baseline levels to measured levels, such as those measured in a care setting such as an ICU, emergency department, hospital, a primary care setting, or other care setting. As just one non-limiting example, this could facilitate risk stratification of patients. Patients at higher risk could be monitored more frequently, put on a different diet, receive prescription changes, and so on. Improved comparisons of baseline creatinine levels to measured levels allows for better understanding of the patient's health and improved diagnoses and care. Thus, implementation of the baseline creatinine determination systems and methods disclosed or otherwise envisioned herein improves patient care and outcomes, and potentially saves lives.

According to an embodiment, the methods and systems described or otherwise envisioned herein comprise numerous applications. For example, the system could be utilized in a pre-hospital setting, as an initial evaluation in an emergency room, in a hospital setting such as an ICU or other in-hospital setting, and in many other settings. The method is applicable to patient care systems, including in point-of-care applications.

Referring to FIG. 2 is a schematic representation of a baseline creatinine determination system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may be located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, training data 262, a trained baseline creatinine determination model 263, and/or reporting instructions 264, among many other possible instructions and/or data.

According to an embodiment, training data 262 comprises data for a plurality of training subjects, which is used to train the baseline creatinine determination model. According to an embodiment, the training dataset comprises, for each of a plurality of training subjects, at least: (i) a set of features for each of the training subjects; and (ii) a measured baseline creatinine value for each of the training subjects. Once obtained, the training dataset may be utilized immediately or stored for future use. The training dataset may be obtained from any database or source of data that comprises the necessary information to train the model.

According to an embodiment, the trained baseline creatinine determination model 263 is any model or algorithm that is trained or configured to determine or estimate a baseline creatinine value for a subject using a set of input features for the subject. The trained baseline creatinine determination model 263 can be trained using any of the methods described or otherwise envisioned herein. The trained baseline creatinine determination model may be stored locally or remotely, and may be a component of the baseline creatinine determination system.

According to an embodiment, reporting instructions 264 direct the system to generate and provide a report or visualization to a user via the user interface 240 of the baseline creatinine determination system 200. The report or visualization comprises, for example, information about the generated baseline creatinine value for the subject. Other information is possible as well, including but not limited to the identity of the patient, patient demographics, diagnosis or treatment information, and a wide variety of other possible information. The information can be provided via the user interface using any method for conveying or displaying information, and the user interface can be any device, interface, or mechanism for providing the conveyed or displayed information. According to an embodiment, the instructions may direct the system to display the information on the user interface or display of the system. The report may be communicated by wired and/or wireless communication to another device. For example, the system may communicate the report to a mobile phone, computer, laptop, wearable device, and/or any other device configured to allow display and/or other communication of the report.

### EXAMPLE

The following is a non-limiting example of a method for training the trained baseline creatinine determination model 263. The model may be trained as described below, or as otherwise envisioned herein. According to an embodiment, the method described below generates an exemplary regression based machine learning model for the prediction of baseline serum creatinine According to an embodiment, the developed model was validated on patients who were admitted in Mayo Clinic intensive care units from 2005-2017. and further validated on the Medical Information Mart for Intensive Care III (MIMIC) cohort, containing records of all ICU admissions from 2001-2012. According to an embodiment, two exemplary individual Gradient Boosting Regression machine learning models are developed for prediction of baseline creatinine, and their output is compared with measured baseline serum creatinine levels and also estimated serum creatinine level back-calculated by the Modification of Diet in Renal Disease (MDRD) equation. According to an embodiment, following ascertainment of eligibility criteria, 44,370 (with baseline<=5.0) patients from Mayo Clinic and 6,112 individuals from the MIMIC-III cohort were enrolled. In comparison with the MDRD equation, both exemplary models in accordance with the present invention performed significantly better (P-Value<0.05) in both cohorts. The initial Mayo cohort I used all had baseline<=0.5, but the MIMIC cohort include those with baseline>5.0. According to an embodiment, using the exemplary machine learning models, baseline serum creatinine could be estimated with higher accuracy in comparison with the MDRD equation and led to more accurate classification of AKI patients. Thus, according to an embodiment, described herein is the development and use of a machine learning system and method along with readily available clinical information in two large cohorts of ICU patients to estimate baseline serum creatinine, resulting in significant decrease in the risk of misclassification of AKI when compared with MDRD-formula back-calculated estimated baseline serum creatinine

According to an embodiment, serum creatinine is the most widely used marker of kidney function in chronic and acute settings. The Kidney Disease Improving Global Outcomes (KDIGO) guidelines for acute kidney injury (AKI) definition is based on acute changes in serum creatinine or urine output. Changes in serum creatinine are calculated as an absolute or relative increase compared to the baseline serum creatinine (BSCr). Therefore, having access to the BSCr is essential to the appropriate classification of patients with AKI. As the availability of measured BSCr is typically between 40-60%, methods for estimating BSCr have been proposed. The majority of studies use one of the following methods: (i) back-calculation of serum creatinine when assuming a GFR of 60 or 75 mL/minute per 1.73 m² using the Modification of Diet in Renal Disease (MDRD) equation, or (ii) using the first or lowest hospital serum creatinine as a baseline. Each of these methods is associated with significant biases regarding the classification of AKI.

The back-calculation of BSCr using the MDRD formula assumes all hospitalized individuals have a baseline GFR of 60 or 75 mL/minute per body surface area. Therefore, back-calculated BSCr using this assumption may incorrectly flag those with chronic kidney disease (CKD) as having AKI, since calculated serum creatinine is lower than the real BSCr. On the other hand, using the first hospital serum creatinine as BSCr tends to underestimate real BSCr in patients who present with community-acquired AKI. In these patients, if their first hospital serum creatinine is considered their baseline; hence, they would be counted as CKD, not AKI. When lowest hospital serum creatinine is used as a surrogate for baseline serum creatinine, there has been a tendency to overestimate AKI incidence presumably due to serum concentration from volume depletion on admission and its resolution by hydration which leads to overdiagnosis of AKI.

### Methods—Participants

Data was collected from the electronic health records of adult patients admitted to ICU at Mayo Clinic in Rochester, MN, from Jan. 1, 2005, to Dec. 31, 2017. All adult, non-pregnant subjects who provided research authorization were included. This study was reviewed and approved by the Mayo Clinic IRB (#07-001380), and the Philips Internal ethical review board. The need for informed consent was waived due to the minimal risk of this retrospective study. Patients with no measured creatinine in 180 to 7 days prior to hospital admission, those who received renal replacement therapy before ICU stay, and had a baseline creatinine above 5 mg/dL were excluded. Patient demographics, weight, and BSCr was also collected. Diagnoses were also abstracted based on the International Classifications of Disease (ICD) 9/10 codes and information on medication history before hospital admission. CKD was defined based on the KDIGO criteria.

### Methods—Model Development

A total of 290 features were available in the electronic health records, including patient demographics (i.e., age, sex, race, weight, height, and Charlson comorbidity index), comorbidities (ICD-9/10 codes grouped into categories), and medication history (nephrotoxic medications grouped into 15 classes). Comorbidities were filtered to retain categories present in at least >1% of the population. Among comorbidities, those that mainly (at least 75% of the time) identified acute conditions were excluded. The remaining 84 features were used as input data to train the model. The dataset was split as 75% of the cohort for training and 25% set aside for testing.

In accordance with exemplary embodiments of the present invention, a gradient boosting regression model was trained using five-fold cross validation, and initially using all 84 features and real BSCr value as the target variable. To optimize training, three of the boosting parameters were tuned: number of estimators (80, 100, 120), maximum depth of individual tree (3,4,5,6), and minimum samples per leaf (1,20,50,80). Values of 100 were settled on for number of estimators, maximum tree depth of 4, and minimum samples per leaf of 20.

To reduce the number of features, the Boruta feature selection algorithm was used, initially starting with all 84 features and with iterative applications reducing the number of features to 9 (model GBR9). Since the MIMIC III validation set was missing some of the features in the Mayo dataset, a model was also trained on the Mayo data using the 70 features that are also available in the MIMIC dataset. After applying Boruta feature selection, there were a total of six features (model GBR6). These six features turned out to be a subset of the features in the GBR9 model.

### Methods—Exemplary Model Evaluation

The gold standard to which these two exemplary models were compared was the real BSCr (defined as the average creatinine charted values 180 days to 7 days prior to hospital admission). Both models in accordance with exemplary embodiments of system and method of the present invention were compared to the gold standard, i.e., the real baseline creatinine (measured between 180 and 7 days prior to hospital admission). In addition, the estimates of baseline creatinine were computed using the Modification in Diet in Renal Disease (MDRD) equation assuming estimated glomerular filtration rate (eGFR) values of 60 mL/minute per 1.73m². The estimate is referred to as MDRD. The MDRD back-calculation equation is:

$$\\ \begin{matrix}BaseCr = {( \frac{eGFR}{186 \times Gender \times Race \times {Age}^{- 0.203}} )}^{- \frac{1}{1.154}} & ( Eq . 1 )\end{matrix}$$

where eGFR (estimated glomerular filtration rate) is 60 mL/minute per 1.73 m², Gender=0.7421 if female and Race=1.21 if black. The values of eGFR were chosen to be 60 mL/minute per 1.73 m² for higher specificity as recommended by KDIGO.

For all three models (the two generated models and the MDRD estimate), the difference between the real baseline creatinine and model estimate was calculated. The ratio of model estimate to real baseline creatinine was also calculated. Four performance metrics were evaluated for both models and the MDRD estimate:
1. Mean absolute error, being the mean of the absolute error between real baseline creatinine and model estimate;
2. Median absolute error, being the median of the absolute error between real baseline creatinine and model estimate;
3. Pearson correlation coefficient, being the correlation between the model estimate and the real baseline creatinine; and
4. Intra-class coefficient, estimated using a two-way mixed model.

Metrics 1-3 were calculated using Python Pandas package version 0.25.1. Metric 4 was calculated using a two-way random-effects model implemented in the irr package using R version 3.2.3. These metrics were calculated for the Mayo and MIMIC cohorts, and the results are shown in Tables 1 and 2, respectively.

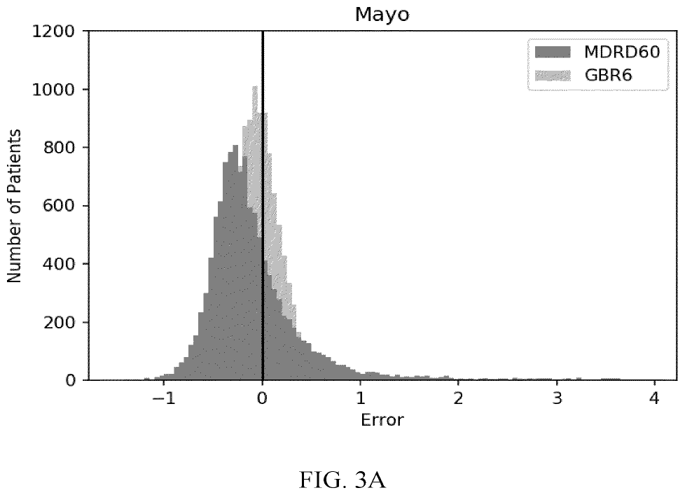

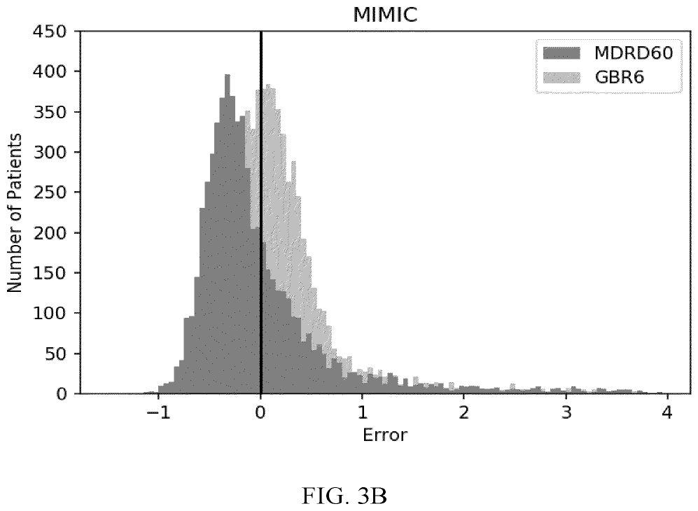

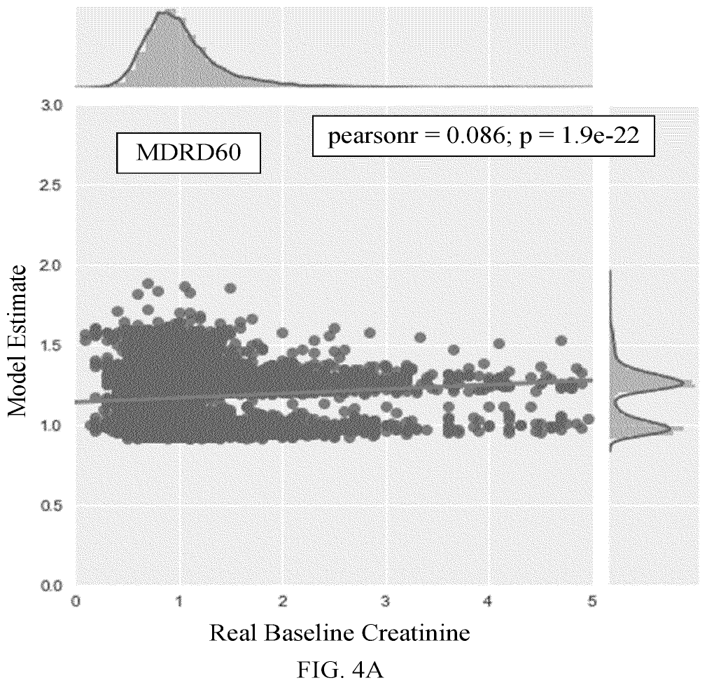

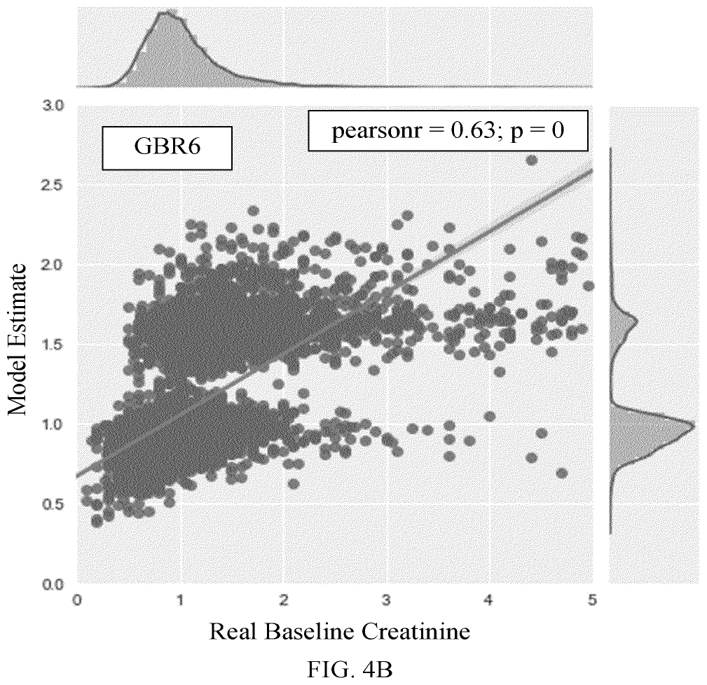

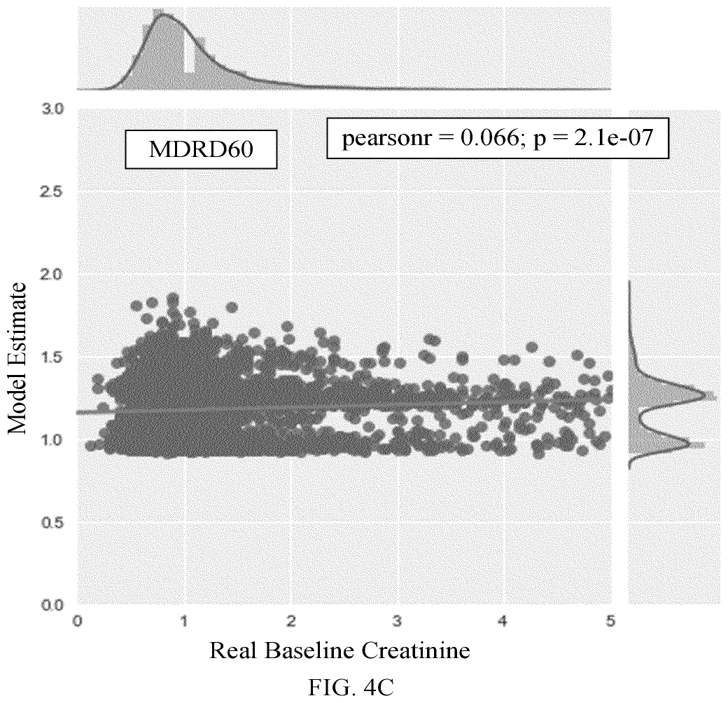

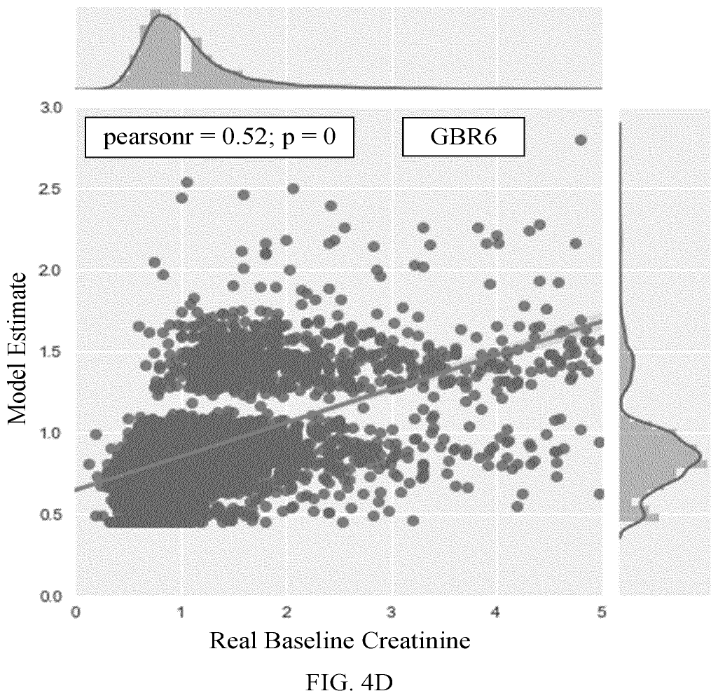

To provide additional information into the exemplary model predictions compared to the real value, the correlation between each model estimate and real baseline creatinine was plotted. The model error (real baseline creatinine—model estimate) was also plotted as a function of the real baseline creatinine These exemplary resulting plots are shown in FIGS. 3A and 3B, as well as FIGS. 4A and 4B.

Referring to FIGS. 3A and 3B, in accordance with an embodiment, are histograms of the error (real baseline-model estimate) for the GBR6 model and MDRD equation for the Mayo Clinic (FIG. 3A) and MIMIC-III (FIG. 3B) cohorts. The GBR6 distribution is centered around 0 (tallest peaks), indicating lower error compared to the MDRD model which is centered to the left of 0.

Referring to FIGS. 4A-4D, in accordance with an embodiment, are comparisons of Mean Absolute Error (MAE) for sub cohorts of real baseline creatinine-Mayo Clinic (FIGS. 4A and 4B), MIMIC-III (FIGS. 4C and 4D). The value of MAE at each point is the value for the subcohort whose upper bound is the point. For example, the value at 3.0 mg/dL represents the subcohort with real BSCr between 2.0-3.0 mg/dL.

TABLE 1
Metrics for the Mayo Cohort

| Mayo: | MDRD | GBR6 | GBR9 |
|---|---|---|---|
| Mean Absolute Error | 0.374 (0.368, | 0.248 (0.243, | 0.246 (0.241, |
| | 0.381) | 0.254) | 0.251) |
| Median Absolute Error | 0.303 (0.296, | 0.170 (0.166, | 0.170 (0.166, |
| | 0.309) | 0.175) | 0.174) |
| Pearson Coefficient | 0.086 (0.070 | 0.628 (0.614, | 0.639 (0.626, |
| | 0.103) | 0.639) | 0.652) |

TABLE 2
Metrics for the MIMIC Cohort

| MIMIC: | MDRD | GBR6 |
|---|---|---|
| Mean Absolute Error | 0.465 (0.452, 0.477) | 0.387 (0.374, 0.400) |
| Median Absolute Error | 0.347 (0.339, 0.354) | 0.221 (0.213, 0.228) |
| Pearson Coefficient | 0.064 (0.039, 0.089) | 0.525 (0.499, 0.551) |
| Intra-class Corr. Coef. | | |

### Methods—Exemplary Sensitivity Analysis

To evaluate the performance of the inventive exemplary model in different cohorts, five cohort strata were defined based on their real baseline creatinine value (mg/dL): <=0.5, (0.5,1], (1.0, 2], (2.0,3.0], (3.0,4.0], (4.0,5.0]. The mean absolute error of the GB6 model and MDRD equation was calculated for the sub-cohort of patients whose real baseline creatinine was in each of the above the strata.

### Methods—Exemplary External Validation: MIMIC III Dataset

These exemplary models in accordance with the present invention were validated on an external dataset, the Medical Information Mart for Intensive Care III (MIMIC-III), a publicly available critical care database from Beth Israel Deaconess Medical Center, Boston containing records of all ICU admissions from 2001-2012. The same eligibility criteria were used to select patients. BSCr was estimated using the five models and MDRD and calculated the performance metrics listed above.

### Results

Among 88,215 Mayo Clinic admissions, 44,370 (50.3%) patients had BSCr measured; in the MIMIC-III database, 6,112 of 37,648 patients (16.2%) had BSCr measurement (Tables 3 and 4). Overall the age and sex distribution were similar, but MIMIC-III cohort had higher mortality and BSCr. For the Mayo Clinic cohort, all the final results are presented from the testing (25%) cohort. Tables 3 and 4 also provide additional information about the differences among those with available measured baseline serum creatinine versus those who did not have serum creatinine measured prior to the index ICU admission.

TABLE 3
Mayo Cohort

| | Mayo (n = 88215) | | | |
|---|---|---|---|---|
| | Base Cr Measured | | Base Cr Not Measured | |
| Number of patients | 44370 | (50.3%) | 43845 | (49.7%) |
| Age at admission (years) | 64.8 | [64.64 64.94] | 59.81 | [59.64 59.98] |
| ICU stay (hours) | 54.9 | [53.67 56.06] | 63.96 | [62.85 65.06] |
| Height (cm) | 169.7 | [169.63 169.82] | 170.44 | [170.34 170.54] |
| Weight (kg) at ICU Admission | 84.69 | [84.47 84.91] | 85.67 | [85.44 85.9] |
| Weight increase (kg) | −0.06 | [−0.1 −0.03] | 0.02 | [−0.01 0.05] |
| Charlson Score | 4.53 | [4.51 4.55] | 3.38 | [3.36 3.4] |
| Death in ICU | 2.13 | [1.99 2.26 | 2.48 | [2.33 2.62] |
| Death in Hospital | 4.59 | [4.4 4.79] | 4.57 | [4.38 4.77] |
| African- American | 1.27 | [1.16 1.37] | 1.6 | [1.49 1.72] |
| Gender | 57.54 | [57.08 58.0] | 57.6 | [57.13 58.06] |
| AKI prior to Admission | 11.79 | [11.49 12.09] | 10.5 | [10.21 10.78] |
| Hypertension with complications and | 17.07 | [16.72 17.42] | 10.37 | [10.08 10.65] |
| secondary hypertension | | | | |
| Chronic kidney disease | 19.08 | [18.71 19.45] | 11.04 | [10.75 11.34] |
| Nephritis; nephrosis; renal sclerosis | 2.41 | [2.27 2.55] | 1.5 | [1.39 1.62] |
| Diabetes mellitus with complications | 9.69 | [9.42 9.97] | 6.71 | [6.47 6.94] |
| Diabetes mellitus without complication | 23.5 | [23.11 23.9] | 18.75 | [18.38 19.11] |
| Congestive heart failure; non-hypertensive | 25.89 | [25.48 26.3] | 18.77 | [18.41 19.14] |
| Deficiency and other anemia | 24.61 | [24.21 25.01] | 18.27 | [17.9 18.63] |
| Other diseases of kidney and ureters | 5.56 | [5.35 5.77] | 4.52 | [4.33 4.71] |
| CNI | 4.41 | [4.22 4.6] | 0.85 | [0.77 0.94] |

TABLE 4
MIMIC Cohort

| | MIMIC (n = 37648) | | | |
|---|---|---|---|---|
| | Base Cr Measured | | Base Cr Not Measured | |
| Number of patients | 6112 | (16.2%) | 31536 | (83.8%) |
| Age at admission (years) | 65.51 | [65.12, 65.89] | 63.78 | [63.58 63.98] |
| ICU stay (hours) | 88.42 | [85.12 91.73] | 92.5 | [91.07 93.93] |
| Height (cm) | 169.64 | [169.18 170.1] | 169.97 | [169.73 170.22] |
| Weight (kg) at ICU Admission | 79.24 | [78.51 79.97] | 80.69 | [80.29 81.08] |
| Weight increase (kg) | -NA- | | -NA- | |
| Charlson Score | -NA- | | -NA- | |
| Death in ICU | 8.26 | [7.57 8.95] | 8.47 | [8.16 8.78] |
| Death in Hospital | 13.11 | [12.26 13.95] | 11.56 | [11.21 11.91] |
| African- American | 9.03 | [8.31 9.75] | 7.24 | [6.95 7.53] |
| Gender | 56.58 | [55.33 57.82] | 56.55 | [56.01 57.1] |
| AKI prior to Admission | 23.54 | [22.48 24.61] | 21.7 | [21.24 22.15] |
| Hypertension with complications and | 11.22 | [10.43 12.02] | 10.13 | [9.8 10.46] |
| secondary hypertension | | | | |
| Chronic kidney disease | 12.37 | [11.54 13.19] | 10.48 | [10.15 10.82] |
| Nephritis; nephrosis; renal sclerosis | 1.51 | [1.2 1.81] | 1.27 | [1.14 1.39] |
| Diabetes mellitus with complications | 8.15 | [7.46 8.83] | 7.4 | [7.12 7.69] |
| Diabetes mellitus without complication | 19.98 | [18.97 20.98] | 18.82 | [18.39 19.25] |
| Congestive heart failure; non-hypertensive | 28.88 | [27.74 30.01] | 23.75 | [23.28 24.22] |
| Deficiency and other anemia | 23.36 | [22.3 24.42] | 20.01 | [19.56 20.45] |
| Other diseases of kidney and ureters | 3.52 | [3.06 3.98] | 2.78 | [2.6 2.97] |

| | | |
|---|---|---|
| CNI | -NA- | -NA- |

### Results—Exemplary Model Performance

Both GBR prediction models in accordance with exemplary embodiments of the present invention had significantly lower error (mean absolute error and median absolute error) and higher correlation (Pearson coefficient and intra-class correlation coefficient) with the measured BSCr than the MDRD estimates (see, for example, Tables 1 and 2). The GBR9 prediction model with nine features was slightly, but not significantly, better than the GBR6 performance model. Since the two prediction models had almost identical performance, the GBR6 model is discussed in greater detail herein, which uses features available in both the Mayo and MIMIC datasets, but is not meant to be limiting.

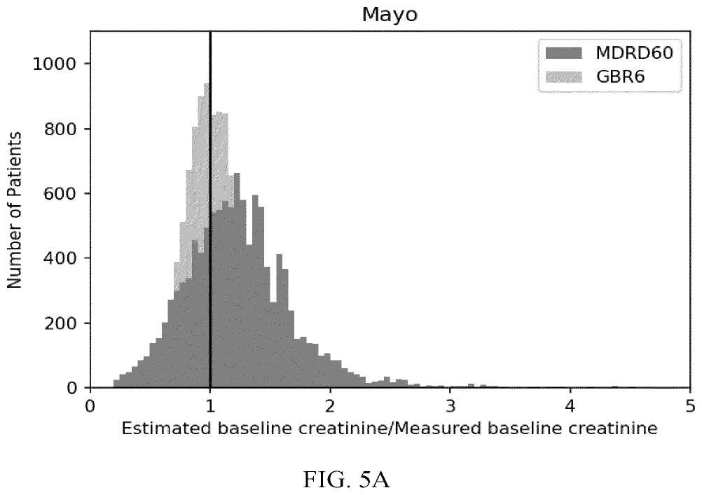

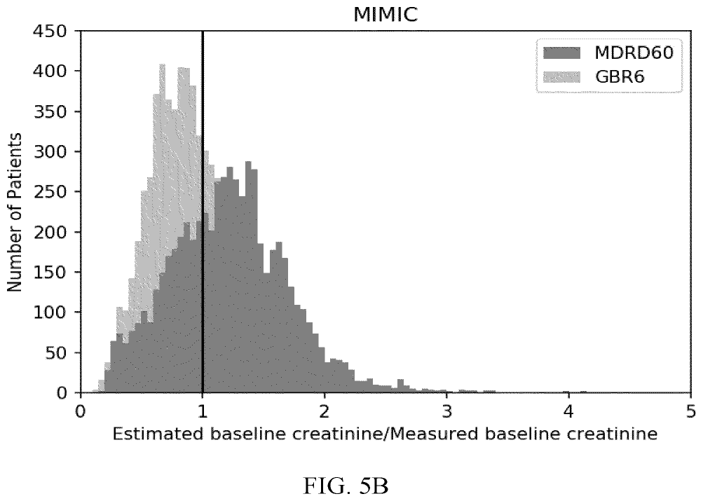

FIGS. 3A and 3B, with FIGS. 5A and 5B, show the distribution of errors in two different ways. FIGS. 3A and 3B shows the histogram of error (measured baseline creatinine—predicted) comparing GBR6 and MDRD-60 models for the two cohorts. The GBR6 model is more centered and has a tighter distribution than the MDRD-60 model (respective means and standard deviations: 0.00, 0.40 vs −0.09, 0.53) (medians: −0.04 vs −0.19) for the Mayo cohort, whereas for the MIMIC cohort MDRD-60 is more centered, although its distribution is still broader (respective means and standard deviations: 0.29, 0.60 vs −0.01, 0.70) (medians: 0.15 vs −0.20).

FIGS. 5A and 5B show the histogram of the ratio of estimated and measured baseline creatinine for the GBR6 model vs. the MDRD-60 model for the Mayo Clinic and MIMIC-III cohorts. Again, the GBR6 model is more centered (at 1.0) and has a tighter distribution than the MDRD-60 model for the Mayo cohort (respective means and standard deviations: 1.09, 0.34 vs 1.25, 0.54) (medians: 1.05 vs 1.21), whereas for the MIMIC cohort both GBR6 and MDRD-60 are off center, but in opposite directions, although, again, the GBR6 distribution is narrower (respective means and standard deviations: 0.88, 0.34 vs 1.23, 0.52) (medians: 0.85 vs 1.21).

The model estimates in accordance with exemplary embodiments of the present invention were better correlated with true BSCr compared to the MDRD estimates (Pearson and Intra-class correlation coefficients in TABLES 1 and 2. MDRD estimates fell in a narrow range of approximately 0.9-1.9 mg/dL and had poor correlation with BSCr (FIGS. 4A and 4B).

### Results—Exemplary Sensitivity Analysis

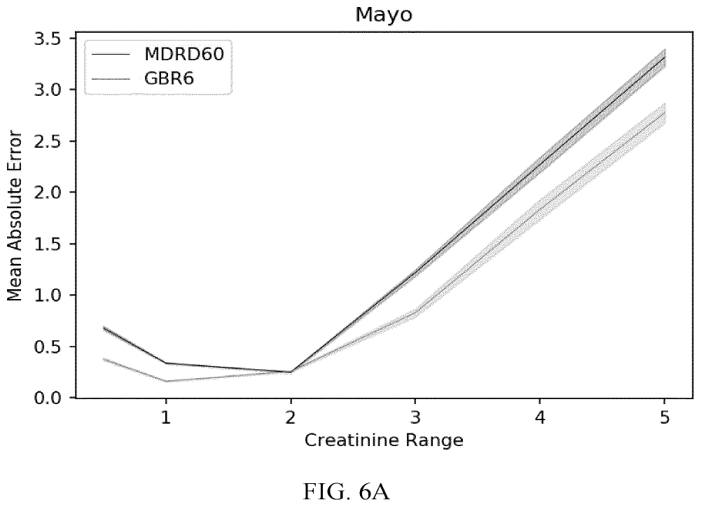

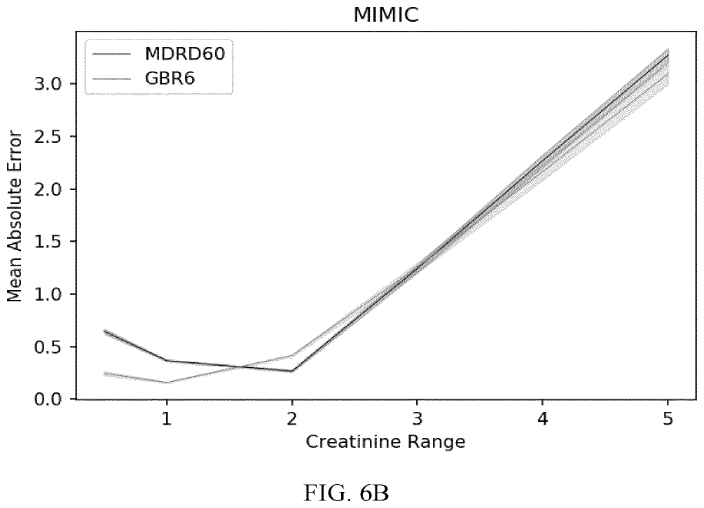

The error is lowest for the strata with BSCr between 0.5-1.0 and 1.0-2.0 mg/dL and sharply rises for BSCr>2.0 mg/dL. For the Mayo cohort, the GBR6 model does significantly better for all the strata except for the 1.0-2.0 mg/dL range of BSCr. In this range, both models have a similar mean absolute error. For the MIMIC cohort, GB6 does better for low values of BSCr (<=0.5), (0.5,1] and high values (>2.0), but significantly worse for the 1.0-2.0 range. These results are shown in FIGS. 6A and 6B. Referring to FIGS. 6A and 6B, therefore, are correlation plots between model estimate and the real baseline. In each subplot the main figure shows the correlation with line of best fit shown in red. The Pearson coefficient and the p-value are shown in top right. The histogram on top shows the distribution of the x-axis (real BSCr) and the histogram on the right shows the distribution of the y-axis. FIG. 6A shows Mayo clinic and FIG. 6B shows MIMIC-III. The top plots are for MDRD vs. real BSCr and bottom plots are for GBR6 vs. real BSCr.

### Results—Exemplary Feature Importance

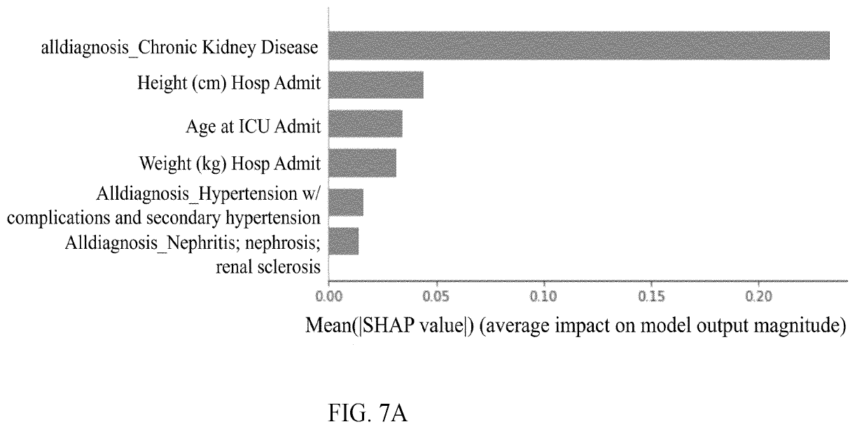

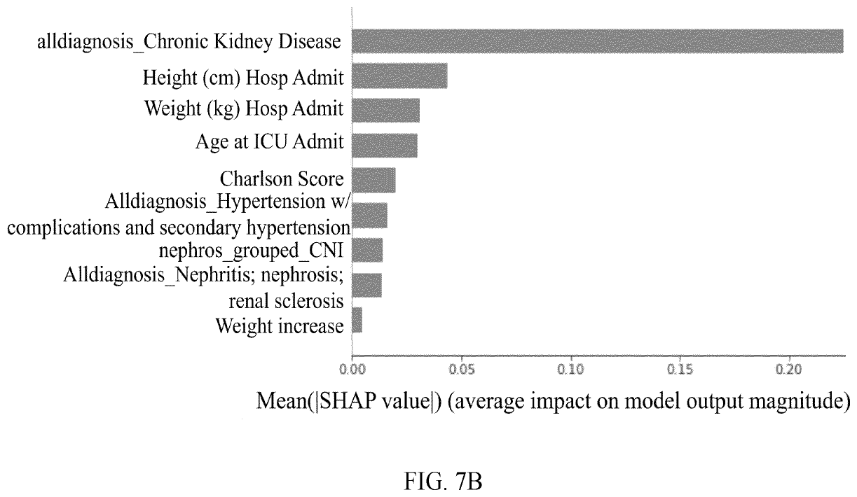

FIGS. 7A and 7B show the feature importance for the GBR6 model (FIG. 7A) and the GBR9 model (FIG. 7B) based on the SHAP (SHapley Additive exPlanations) values. The most important features in estimating BSCr were the presence of CKD, Height, Age and Weight. These were also the top four features in the GBR9 model. Diagnosis of hypertension and diagnosis of nephritis were other 2 diagnoses selected by both models. The GB-9 model had weight increase (from hospital admission to ICU admission) and history of taking calcineurin inhibitors (CNI) as additional features.

Recently, there have been several published models to predict AKI among ICU and hospital patients using machine learning technologies. The idea of using machine learning technology to predict BSCr by available clinical data at the time of hospital or ICU admission is novel, and is described herein. In contrast, a model based on gradient boosting machine learning methods in accordance with exemplary embodiments perform superior to the back-calculation of serum creatinine-based on estimated GFR.

Having access to the BSCr level can be critical in identifying and staging AKI. Using measured serum creatinine prior to ICU admission may be considered the standard for the determination of BSCr level. One study used a panel of nephrologist experts to define the reference standard of BSCr among 379 patients and reported that the average outpatient serum creatinine measurements between 180-7 days prior to ICU admission had the highest intra-class correlation with their reference standard. In another study, investigators reported that the minimum value of preadmission serum creatinine increased the sensitivity of the AKI definition and had superior performance in prediction of 60-day mortality rate when compared with the most recent measured serum creatinine

A major problem generally occurs when there is no measured serum creatinine available to estimate baseline kidney function. Current estimation strategies typically are all associated with significant flaws. In a study of 4,863 hospitalized patients with measured serum creatinine prior to admission, using the lowest or first post-admission creatinine or the back-calculated serum creatinine using MDRD formula with eGFR of 75 mL/minute per 1.73 m² resulted in incorrectly estimated AKI incidence (sensitivity ˜40%) or inflated AKI incidence (specificity ˜80%), respectively. Another group of investigators observed that using the MDRD formula back-calculation for estimating BSCr level impacts the incidence of AKI at lower stages. Pickering et al. compared estimated BSCr with back-calculation of MDRD formula for GFR of 75 and 100 mL/minute per 1.73 m², random assignment of BSCr, and lowest hospital serum creatinine in the classification of AKI among 224 hospitalized patients. They found that using back-calculation overestimated AKI incidence while using the lowest hospital serum creatinine or random assignment accurately identified the proportion of patients with AKI. Some authors have reported the superiority of the first hospital serum creatinine over back-calculation. It seems MDRD back-calculation overall does perform better if it is used among patients without CKD. In addition to the current eGFR formula, including MDRD, a recent study presented a new linear-regression-based formula and demonstrated that it improves the current estimation models in the classification of AKI. While this model was superior to other formulae for back-calculation of serum creatinine, its performance in comparison with measured serum creatinine remained suboptimal.

In accordance with exemplary embodiments of system and method of the present invention, two gradient-boosting based models to predict baseline creatinine were built: a model based on the 84 features in the Mayo database, ending up with a nine feature model, and a model based on the subset of 70 features in the MIMIC database, ending up with a six feature model (which were a subset of the nine features in the other model). The six features in both models were: the presence of chronic kidney disease, age at ICU admit, weight and height at hospital admit, hypertension, and diagnosis of nephritis, nephrosis, or renal sclerosis. The three additional features in the nine feature model were: Charlson score, weight increase, and nephros_grouped_CNI (one of 15 classes of nephrotoxic medications). These features were not available in the MIMIC database. Both models performed about the same.

Both machine learning models in accordance with exemplary embodiments of the present invention had very similar performance to each other in the Mayo cohort and better performance than the MDRD, with lower absolute error and higher correlation to the real BSCr in both cohorts. The results above show the histograms of the difference and ratio of predicted and real BSCr. In both cases, in the Mayo cohort, the GBR6 predictions are tightly centered around 0 and 1, indicating more accurate predictions and less skew. Although the absolute error of the models in the MIMIC-III cohort was higher than the Mayo Clinic cohort, this was still lower than the corresponding absolute error from the MDRD estimate. In the MIMIC cohort, the difference between GBR6 predicted BSCr and real BSCr is centered around 0, the ratio is not, indicating that although the error is lower, the correlation is poorer. This is likely due to the fact the models were trained on the Mayo cohort.

Accuracy was also evaluated on different sub-strata and it was found that the GBR6 did significantly for the low BSCr strata (<=0.5 and (0.5,1.0]) than the MDRD estimates for both the Mayo and MIMIC cohorts. The (0.5,1.0) is the largest strata, making up about half the patients. However, for the second biggest strata, (1.0,2.0], GBR6 performed about the same as MDRD for the Mayo cohort and significantly worse for the MIMIC cohort. At higher values of BSCr, the GBR6 model predictions were better than MDRD in both Mayo and MIMIC cohorts. These findings can be explained by looking at the correlation plots. The MDRD estimated BSCr values are typically within (1-1.5 mg/dL). A majority of the real BSCr values lie between (0.5-2.0 mg/dL). Therefore the overlap between these estimated and real values are higher in this range. However, since the GBR6 model is a more personalized model, the predictions are well-distributed over the range of the BSCr and it tends to perform better in the extreme cases.

Unexpectedly, of the three features that make up MDRD, age, gender and race, the exemplary GBR models in accordance with exemplary embodiments of system and methods described or otherwise envisioned herein which only used age had better results than heretofore known models, system and methods. In the case of race, this may because there were so nonwhite patients in the training set. This could explain why the GBR6 on the MIMIC cohort, which has a much larger share of black patients, was shifted off center as shown above, even though it still retained a fairly narrow distribution. As far as gender is concerned, it is possible that height stood in as a surrogate for gender, and also interacted with weight, to provide a more accurate measure of a patient being over or under weight (much like BMI).

It should be noted that in exemplary embodiments, estimates may be affected by the availability of input feature values. For example, in settings where some of these exemplary input feature values are unavailable, exemplary embodiments of the present invention may not perform as well. For best results, large datasets of patients may be used for development, training and validation of models in accordance with exemplary embodiments of the present invention, and also the implementation of internal and external validation along with multiple sensitivity analyses.

Through developing, training and both internally and externally validating exemplary embodiments of system and method for machine-learning model to estimate BSCr, the methods and systems presented herein has been shown to be superior to the currently heretofore known tools to estimate BSCr. In accordance with exemplary embodiments, an extensive dataset of ICU patients with available BSCr was utilized as a reference standard to train and validate a model. External validation of the model in accordance with exemplary embodiments using the MIMIC-III data set is an additional value of the presented exemplary BSCr estimation model. While the methods and systems have been described in detail with reference to the datasets defined herein in the description above and with reference to the figures below, one having ordinary skill in the art shall appreciate that exemplary embodiments of the present invention could be used in/with any dataset that is available in different healthcare systems. Moreover, one having ordinary skill in the art shall appreciate that the disclosure provided herein is of exemplary embodiments of the present invention, and that modifications could be made that, while not explicitly disclosed herein, have been contemplated by the inventors and are considered as having been disclosed and described as a part of the embodiments described or otherwise envisioned herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for determining a baseline creatinine value for a subject, comprising:
obtaining a set of features about the subject;
analyzing, using a trained baseline creatinine determination model, the obtained set of features to generate a baseline creatinine value for the subject;
reporting, via a user interface, the generated baseline creatinine value for the subject.

2. The method of claim 1, wherein the set of features comprises: (i) chronic kidney disease status; (ii) age of the subject; (iii) weight of the subject; (iv) height of the subject; (v) hypertension status; and (vi) nephritis, nephrosis, and/or renal sclerosis status.

3. The method of claim 2, wherein the set of features further comprises: (vii) Charlson comorbidity index; (viii) weight increase from hospital admission to ICU admission; and (ix) history of calcineurin inhibitor intake.

4. The method of claim 1, further comprising training the baseline creatinine determination model, comprising:
obtaining training data comprising data for a plurality of training subjects, the data comprising: (i) a set of features for each of the training subjects; and (ii) a measured baseline creatinine value for each of the training subjects;
training the baseline creatinine determination model using the training dataset to generate a trained baseline creatinine determination model, wherein training comprises identifying a subset of the set of features that generates a generated baseline creatinine value for a training subject that best correlates with the measured baseline creatinine value for that subject, wherein the identified subset of features comprises input features for the trained baseline creatinine determination model; and
storing the trained baseline creatinine determination model.

5. The method of claim 4, wherein the set of features for each of the training subjects comprises at least (i) chronic kidney disease status; (ii) age of the training subject; (iii) weight of the training subject; (iv) height of the training subject; (v) hypertension status; and (vi) nephritis, nephrosis, and/or renal sclerosis status.

6. The method of claim 1, wherein the trained baseline creatinine determination model is a gradient boosting regression model.

7. The method of claim 1, wherein reporting via the user interface further comprises providing one or more of demographic information about the patient and information about the set of features.

8. The method of claim 1, further comprising administering a treatment to the subject based at least in part on the reported generated baseline creatinine value for the subject.

9. A system for determining a baseline creatinine value for a subject, comprising:
a set of features about the subject;
a trained baseline creatinine determination model;
a processor configured to analyze, using the trained baseline creatinine determination model, the obtained set of features to generate a baseline creatinine value for the subject; and
a user interface configured to report the generated baseline creatinine value for the subject.

10. The system of claim 9, wherein the set of features comprises: (i) chronic kidney disease status; (ii) age of the subject; (iii) weight of the subject; (iv) height of the subject; (v) hypertension status; and (vi) nephritis, nephrosis, and/or renal sclerosis status.

11. The system of claim 10, wherein the set of features further comprises: (vii) Charlson comorbidity index; (viii) weight increase from hospital admission to ICU admission; and (ix) history of calcineurin inhibitor intake.

12. The system of claim 9, further comprising:
training data comprising data for a plurality of training subjects, the data comprising: (i) a set of features for each of the training subjects; and (ii) a measured baseline creatinine value for each of the training subjects;
wherein the processor is further configured to train the baseline creatinine determination model using the training dataset to generate a trained baseline creatinine determination model, wherein training comprises identifying a subset of the set of features that generates a generated baseline creatinine value for a training subject that best correlates with the measured baseline creatinine value for that subject, wherein the identified subset of features comprises input features for the trained baseline creatinine determination model.

13. The system of claim 12, wherein the set of features for each of the training subjects comprises at least (i) chronic kidney disease status; (ii) age of the training subject; (iii) weight of the training subject; (iv) height of the training subject; (v) hypertension status; and (vi) nephritis, nephrosis, and/or renal sclerosis status.

14. The system of claim 9, wherein the trained baseline creatinine determination model is a gradient boosting regression model.

15. The system of claim 9, wherein reporting via the user interface further comprises providing one or more of demographic information about the patient and information about the set of features.
